(19) United States
(12) Patent Application Publication
(10) Pub. No.: US 2021/0059507 A1
(43) Pub. Date: Mar. 4, 2021

(54) INSERTION APPARATUS AND DISTAL END COVER OF INSERTION APPARATUS

(71) Applicant: OLYMPUS CORPORATION, Tokyo (JP)

(72) Inventor: Koji YAMAYA, Tokyo (JP)

(73) Assignee: OLYMPUS CORPORATION, Tokyo (JP)

(21) Appl. No.: 17/097,156

(22) Filed: Nov. 13, 2020

Related U.S. Application Data
(63) Continuation of application No. PCT/JP2019/007374, filed on Feb. 26, 2019.

(30) Foreign Application Priority Data
May 21, 2018 (JP) 2018-096957

(51) Int. Cl. A61B 1/00; A61B 1/018

(52) U.S. Cl. CPC A61B 1/00098; A61B 1/018; A61B 1/00101; A61B 1/00137

(57) ABSTRACT

An insertion apparatus includes first and second distal end portion-side bearing portions at a distal end portion (distal end configuration part), and when a distal end cover main body is attached to the distal end portion, supports a shaft portion rotatably supporting a raising base with respect to a distal end cover main body (distal end cover) by the first and second distal end portion-side bearing portions.

### CROSS REFERENCE TO RELATED APPLICATION

This application is a continuation application of PCT/JP2019/007374 filed on Feb. 26, 2019 and claims benefit of Japanese Application No. 2018-096957 filed in Japan on May 21, 2018, the entire contents of which are incorporated herein by this reference.

### BACKGROUND OF THE INVENTION

### 1. Field of the Invention

The present invention relates to an insertion apparatus in which a distal end cover including a raising member for raising a treatment instrument is detachably attached to insertion equipment, and a distal end cover of the insertion apparatus.

### 2. Description of the Related Art

In the medical field, conventionally, an endoscope has been widely used as an insertion apparatus capable of observing organs and the like from a body cavity by insertion of an elongated insertion portion into the body cavity. In such a type of endoscope, various treatments in the body cavity can be performed in a state where a treatment instrument inserted through a treatment instrument insertion opening provided in an operation portion protrudes from a distal end portion of the insertion portion.

In the treatment using such a treatment instrument, a raising base (forceps elevator) is widely used to change a distal end of the treatment instrument protruding into the body cavity in a desired direction by an operation on a hand side.

In order to facilitate cleaning of the endoscope, a configuration has been proposed in recent years in which such a type of raising base is provided on a distal end cover configured to be attachable to and detachable from the distal end portion of the endoscope.

For example, a technique is disclosed in Japanese Patent Application Laid-Open Publication No. H6-315458 in which a raising base is provided via a raising shaft (support shaft) to be rotatable within a predetermined angle range with respect to a slope portion body of a distal end attachment/detachment portion (distal end cover) that is attachable to and detachable from a distal end portion.

### SUMMARY OF THE INVENTION

An insertion apparatus according to an aspect of the present invention includes: an insertion portion configured to be inserted into a subject and configured to allow insertion of another insertion apparatus into the insertion portion; a distal end cover detachably arranged at a distal end portion of the insertion portion; a raising member provided on the distal end cover to change an advancing direction of the other insertion apparatus; a shaft member provided on the distal end cover to rotatably support the raising member with respect to the distal end cover; and a support portion provided at the distal end portion and configured to abut on the shaft member to support the shaft member when the distal end cover is attached to the distal end portion.

A distal end cover of an insertion apparatus according to an aspect of the present invention includes: a cover main body attached to a distal end portion of an insertion portion of the insertion apparatus; a raising member provided on the cover main body to change an advancing direction of another insertion apparatus inserted into the insertion portion; and a shaft member provided on the cover main body to rotatably support the raising member with respect to the cover main body, in which the shaft member is supported by a bearing provided at the distal end portion of the insertion portion of the insertion apparatus.

### BRIEF DESCRIPTION OF THE DRAWINGS

FIG. 1 is a side view showing a configuration of an endoscope apparatus according to a first embodiment of the present invention;

FIG. 2 is a side view showing a state where a distal end cover device is removed from an endoscope according to the first embodiment of the present invention;

FIG. 3 is a perspective view showing a distal end cover before being attached to a distal end portion according to the first embodiment of the present invention;

FIG. 4 is a perspective view showing the distal end cover attached to the distal end portion according to the first embodiment of the present invention;

FIG. 5 is a cross-sectional view of the distal end portion and the distal end cover according to the first embodiment of the present invention;

FIG. 6 is a cross-sectional view mainly showing a connection portion between a distal end cover main body and a guide sheath according to the first embodiment of the present invention;

FIG. 7 is a plan view of the distal end cover main body according to the first embodiment of the present invention;

FIG. 8 is a perspective view showing a state where the distal end cover is removed from the distal end portion according to the first embodiment of the present invention;

FIG. 9 is a perspective view showing a relation between a shaft portion and a bearing portion before the distal end cover is attached to the distal end portion according to the first embodiment of the present invention;

FIG. 10 is a perspective view showing a relation between the shaft portion and a distal end portion-side bearing portion when the distal end cover is attached to the distal end portion according to the first embodiment of the present invention;

FIG. 11 is a vertical cross-sectional view showing main parts of the distal end cover according to the first embodiment of the present invention;

FIG. 12 is a perspective view showing a relation between the shaft portion and a cover-side bearing portion according to the first embodiment of the present invention;

FIG. 13 is a cross-sectional view taken along a line XIII-XIII in FIG. 11 according to the first embodiment of the present invention;

FIG. 14 is a cross-sectional view of main parts of a distal end portion and a distal end cover according to a first modification;

FIG. 15 is a perspective view showing a distal end cover before being attached to a distal end portion according to a second modification;

FIG. 16 is a perspective view showing a distal end cover before being attached to a distal end portion according to a third modification;

FIG. 17 is a perspective view showing a distal end cover before being attached to a distal end portion according to a fourth modification;

FIG. 18 is a perspective view showing the distal end cover attached to the distal end portion according to the fourth modification;

FIG. 19 is a perspective view showing a distal end cover before being attached to a distal end portion according to a second embodiment of the present invention;

FIG. 20 is a perspective view showing the distal end cover attached to the distal end portion according to the second embodiment of the present invention;

FIG. 21 is a perspective view showing a relation between a shaft portion and a distal end portion-side bearing portion when the distal end cover is attached to the distal end portion according to the second embodiment of the present invention;

FIG. 22 is an exploded perspective view showing main parts of a distal end cover and a distal end portion according to a third embodiment of the present invention;

FIG. 23 is a perspective view of a raising base according to the third embodiment of the present invention;

FIG. 24 is a perspective view showing a relation between the raising base and a drive shaft according to the third embodiment of the present invention;

FIG. 25 is a cross-sectional view of main parts of the distal end portion according to the third embodiment of the present invention; and

FIG. 26 is a cross-sectional view of the main parts of the distal end portion to which the distal end cover is attached according to the third embodiment of the present invention.

### DETAILED DESCRIPTION OF THE PREFERRED EMBODIMENTS

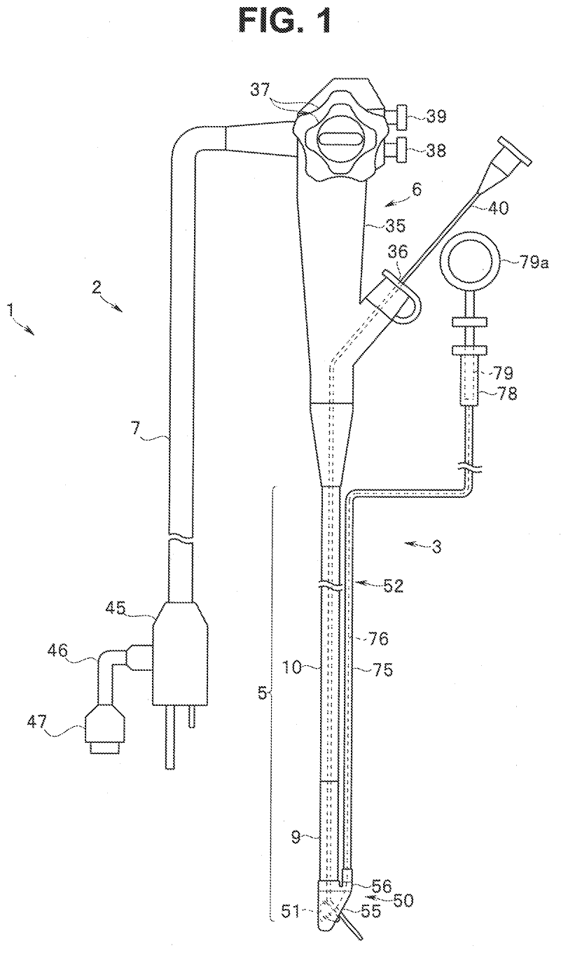

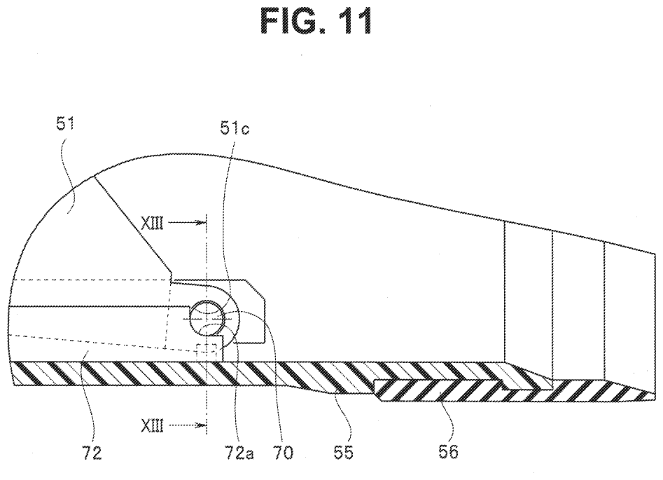

A first embodiment of the present invention will be described below with reference to FIGS. 1 to 13.

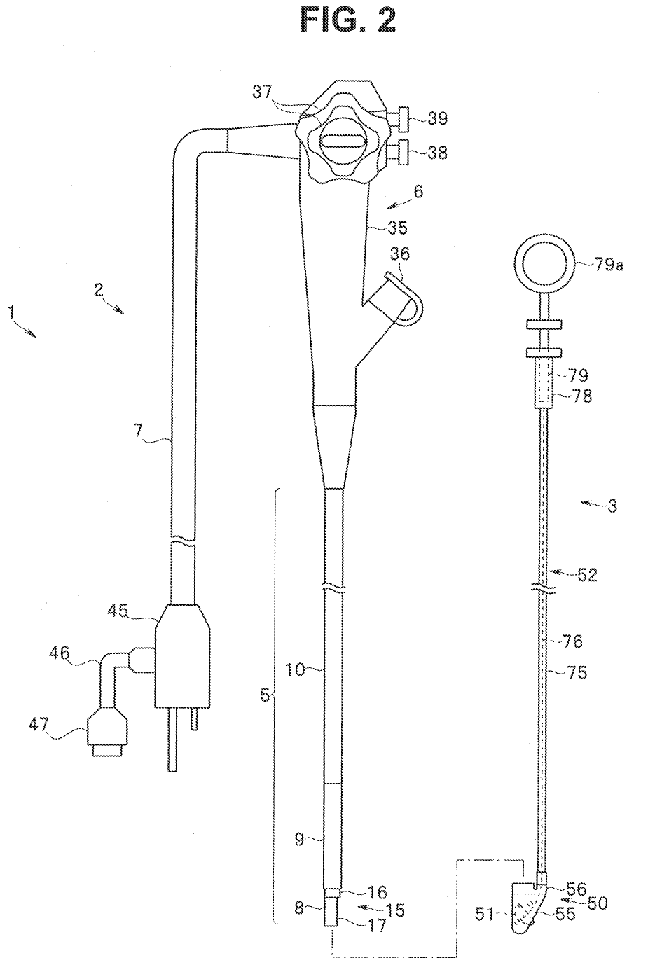

As shown in FIGS. 1 and 2, an endoscope apparatus 1 of the present embodiment includes an endoscope 2 and a distal end cover device 3 detachably attached to the endoscope 2.

The endoscope 2 includes an insertion portion 5, an operation portion 6, and a universal cable 7. The insertion portion 5 is an elongated long member, and includes a distal end portion 8, a bending portion 9, and a flexible tube portion 10 which are continuously provided in order from a distal end side.

The distal end portion 8 includes a distal end configuration part 15 formed of a metal such as stainless. The distal end configuration part 15 includes a base portion 16 and an observation protrusion portion 17 protruding in a direction of an insertion axis O from a distal end of the base portion 16. A ring-shaped insulation member 20 having insulation properties is provided on an outer periphery of a proximal end side of the distal end configuration part 15, and an envelope 21, a distal end of which is fixed to the distal end configuration part 15 by a thread-wound bonding portion 21a is continuously provided at a proximal end compared with the insulation member 20.

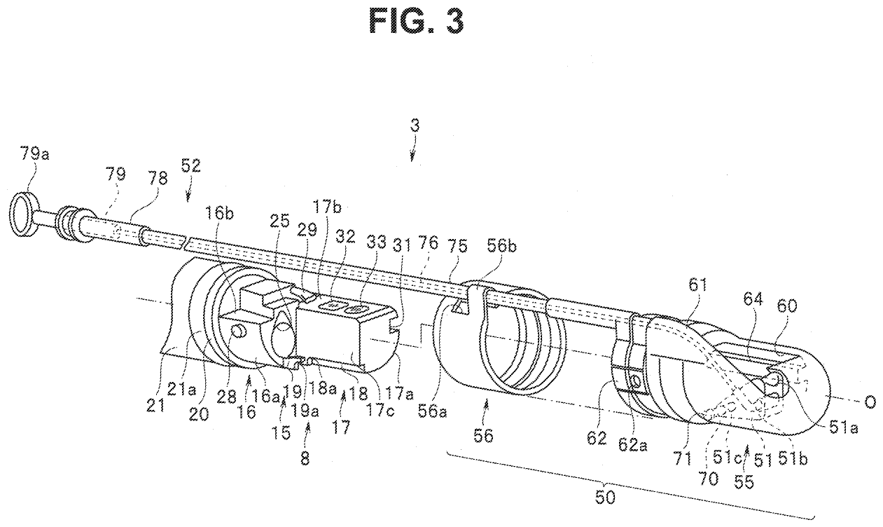

For example, as shown in FIG. 3, the base portion 16 is formed to have an approximate shape in which approximately half of a cylinder is cut out (that is, a substantially semi-cylindrical shape). Thus, the base portion 16 includes a circular arc surface portion 16a and a planar surface portion 16b on an outer periphery.

In addition, the observation protrusion portion 17 is formed to have an approximate shape in which substantially three-quarters of a cylinder arranged coaxially with the base portion 16 are cut out (that is, a substantially quarterly cylindrical shape). Thus, the observation protrusion portion 17 includes, on an outer periphery, a circular arc surface portion 17a, a first planar surface portion 17b which is, for example, parallel to the planar surface portion 16b of the base portion 16, and a second planar surface portion 17c which is, for example, orthogonal to the first planar surface portion 17b.

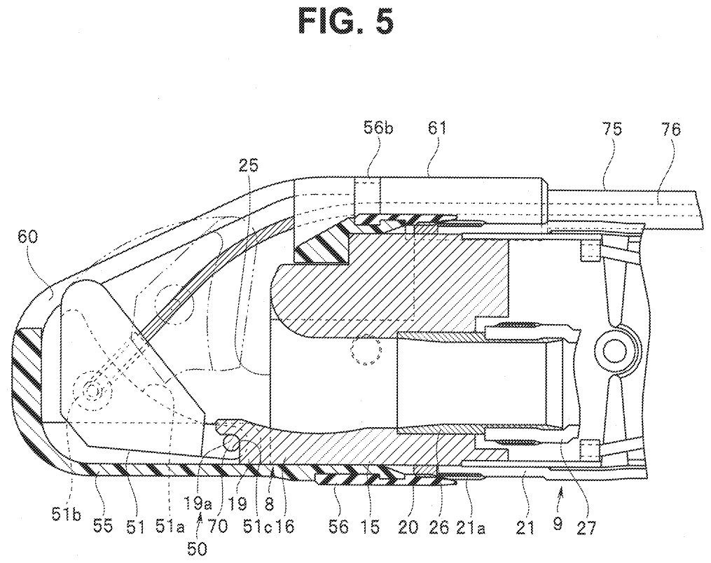

On the end surface of the base portion 16, a treatment instrument protrusion port 25 is provided at a position adjacent to the second planar surface portion 17c of the observation protrusion portion 17. As shown in FIG. 5, a coupling pipe 26 is coupled to the treatment instrument protrusion port 25 on the proximal end side of the distal end configuration part 15, and a distal end side of a treatment instrument channel 27 inserted into the insertion portion 5 is coupled to the coupling pipe 26.

Further, an engagement pin 28 protruding in a direction orthogonal to the insertion axis O is provided on the circular arc surface portion 16a of the base portion 16.

Further, a nozzle 29 is protrusively provided at the base portion 16 to feed air or water toward the first planar surface portion 17b formed on the observation protrusion portion 17.

A key groove 31 extending along the insertion axis O is provided on the circular arc surface portion 17a of the observation protrusion portion 17.

The first planar surface portion 17b of the observation protrusion portion 17 is provided with an observation window 32 of an image pickup unit not shown and an illumination window 33 of an illumination unit not shown. With the observation window 32 being arranged as described above, the endoscope 2 of the present embodiment is a so-called side-viewing endoscope having an observation field of view in a direction intersecting the insertion axis O.

The bending portion 9 is configured to be bendable in, for example, four directions (up, down, left, and right directions) orthogonal to the insertion axis O. The flexible tube portion 10 is a long tubular member having flexibility. The up and down directions and the left and right directions described above are defined for convenience with respect to the insertion portion 5, and each of the directions is defined based on, for example, the direction (up direction) in which the observation window 32 is provided.

The operation portion 6 is continuously provided at a proximal end of the flexible tube portion 10. A grasping portion 35 is set in the middle of the operation portion 6 such that an operator or the like grasps.

A treatment instrument insertion opening 36 is provided on a distal end side of the operation portion 6 compared with the grasping portion 35. The treatment instrument insertion opening 36 is coupled to a proximal end side of the treatment instrument channel 27 inside the operation portion 6. Thus, for example, as shown in FIG. 1, a treatment instrument 40 as another insertion apparatus inserted into the treatment instrument insertion opening 36 can protrude from the treatment instrument protrusion port 25 of the distal end portion 8.

In addition, for example, an angle knob 37, an air/water feeding button 38, and a suction button 39 are provided on a proximal end side of the operation portion 6 compared with the grasping portion 35, the angle knob 37 being configured to bend the bending portion 9, the air/water feeding button 38 being configured to feed air or water from the nozzle 29 provided at the distal end portion 8, the suction button 39 being configured to suck a suction target object existing in a subject from the treatment instrument protrusion port 25.

The universal cable 7 extends from a side surface on the proximal end side of the operation portion 6. An endoscope connector 45 connected to a light source apparatus (not shown) is provided at an end portion of the universal cable 7. A signal transmission cable 46 is provided to extend from a lateral part of the endoscope connector 45. An electric connector 47 connected to a video processor (not shown) is provided at the other end side of the signal transmission cable 46.

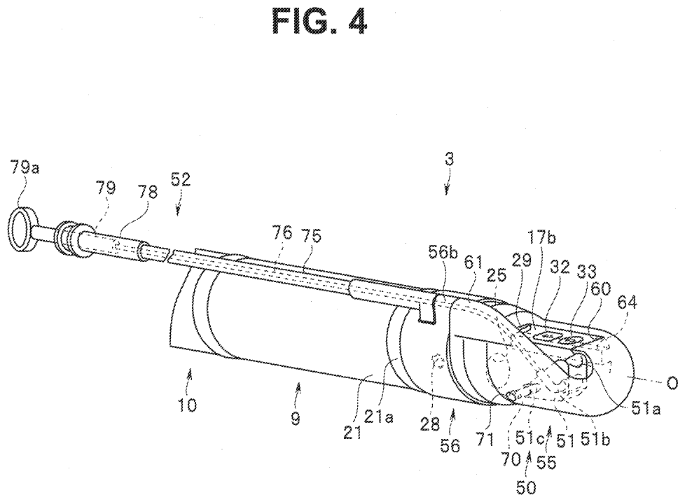

As shown in FIGS. 3 and 4, the distal end cover device 3 includes a distal end cover 50 detachably attached to the distal end portion 8, a raising base 51 provided as a movement member (raising member) inside the distal end cover 50, and a wire mechanism portion 52 that operates the raising base 51 from a hand side.

The distal end cover 50 includes a distal end cover main body 55 as a first cover and a ring cover 56 as a second cover.

The distal end cover main body 55 is formed of a material such as plastic having rigid and insulation properties. The distal end cover main body 55 has a substantially cylindrical shape with a distal end closed in a hemispherical shape, and can be detachably fixed to the outer periphery of the distal end portion 8 to mainly cover the distal end configuration part 15.

The distal end cover main body 55 is provided with an opening portion 60 through which the observation window 32 and the illumination window 33 are exposed and the treatment instrument 40 protruding from the treatment instrument protrusion port 25 is led out.

In the distal end cover main body 55, a sheath connection portion 61 is protrusively provided on one side of the opening portion 60, as a fixing portion used to connect a distal end of a guide sheath 75 configuring a wire mechanism portion 52, which will be described below.

In the distal end cover main body 55, an engagement piece 62 is formed on one side of the opening portion 60 and the sheath connection portion 61, and on a proximal end side of the distal end cover main body 55. The engagement piece 62 can be elastically deformed in an outer diameter direction of the distal end cover main body 55, and an engagement hole 62a is provided in the engagement piece 62, as an engagement portion that can be engaged with the engagement pin 28 protruding from the base portion 16 of the distal end configuration part 15.

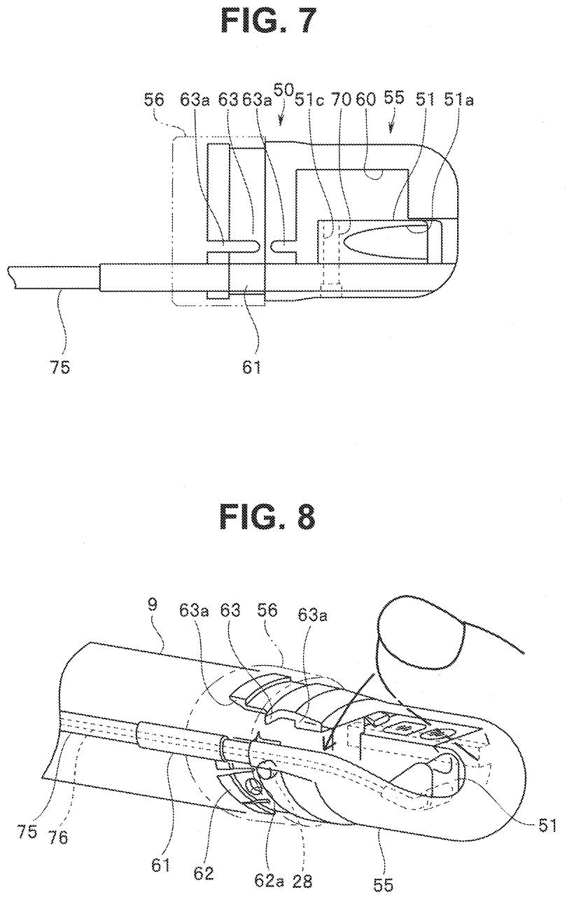

In the distal end cover main body 55, as shown in FIG. 7, a slit 63a is provided on the other side of the sheath connection portion 61, and on a proximal end side of the opening portion 60. With the slit 63a, a fragile portion 63 is set in the distal end cover main body 55 such that the distal end cover main body 55 can break up in a diameter expanding direction.

Further, a key 64 is provided inside the distal end cover main body 55 to fit into the key groove 31. Then, when the key 64 fits into the key groove 31, the distal end cover main body 55 can be positioned at an appropriate position with respect to the distal end configuration part 15.

The ring cover 56 is formed of a material such as silicone rubber having elastic and insulation properties, and is a ring-shaped member having a diameter smaller than an outer diameter of the insulation member 20 of the distal end portion 8 and an outer diameter of the distal end cover main body 55.

The ring cover 56 is elastically deformed in the diameter expanding direction, and thus can be arranged at a position to integrally cover the proximal end side region of the distal end portion 8 provided with the insulation member 20 and the proximal end side region of the distal end cover main body 55. Then, the ring cover 56 can be brought into close contact with the outer peripheries of the distal end portion 8 (insulation member 20) and the distal end cover main body 55 over the entire circumference due to an elastic restoring force.

The ring cover 56 of the present embodiment is provided with a hole portion 56a and a bridge portion 56b to prevent interference with the sheath connection portion 61.

For example, as shown in FIG. 5, the raising base 51 is composed of a member made of metal having a substantially triangular shape in a side view.

A guide groove 51a is provided on an upper surface of the raising base 51 to have a predetermined elevation angle from the proximal end side toward the distal end side.

In addition, a wire connection portion 51b, which connects with an operation wire 76 of a wire mechanism portion 52 to be described below, is provided on a lateral part of the distal end side of the raising base 51.

Further, a shaft insertion hole 51c is provided at the proximal end portion of the raising base 51, and a shaft portion 70 as a coupling member (shaft member) is inserted into the shaft insertion hole 51c. Then, the shaft portion 70 is supported by the distal end cover main body 55, so that the raising base 51 is coupled to the distal end cover main body 55 in a swingable (displaceable) manner.

More specifically, for example, as shown in FIG. 13, a case-side bearing hole 71 is provided on one side of the distal end cover main body 55 to penetrate inside and outside the distal end cover main body 55.

Further, a case-side bearing protrusion 72 is provided inside the distal end cover main body 55, and a case-side bearing portion 72a is provided coaxially with the case-side bearing hole 71 in the case-side bearing protrusion 72.

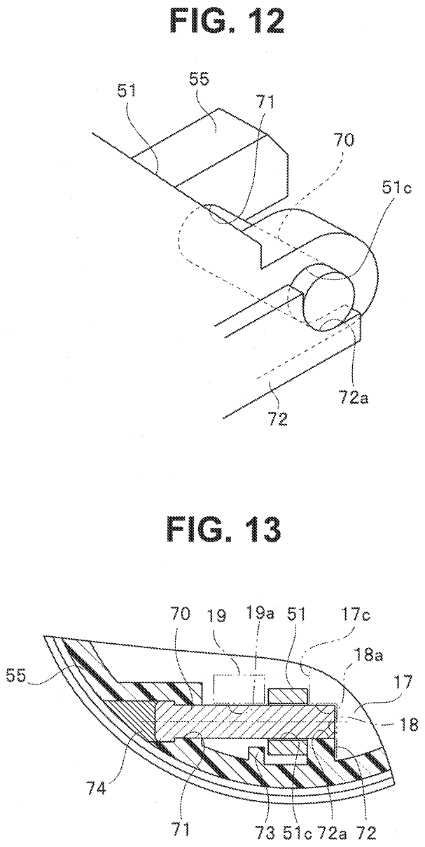

Here, as shown in FIGS. 11 and 12, the case-side bearing portion 72a of the present embodiment has a partial circular arc surface formed by cutting a part of an upper side of the case-side bearing protrusion 72.

Then, the shaft portion 70 is supported by insertion into the case-side bearing hole 71, and the shaft portion 70 is supported by abutting with the case-side bearing portion 72a, so that the raising base 51 is pivotally supported in a swingable manner inside the distal end cover main body 55. For example, as shown in FIG. 13, the case-side bearing hole 71 is sealed with a filling agent 74 such as an adhesive in the state where the shaft portion 70 is inserted.

Further, a restriction protrusion 73 is provided inside the distal end cover main body 55 such that the proximal end side of the raising base 51 is sandwiched between the restriction protrusion 73 and the case-side bearing protrusion 72 to restrict movement of the raising base 51 in an axial direction of the shaft portion 70.

Here, the case-side bearing protrusion 72 and the restriction protrusion 73 are provided at a position where the case-side bearing protrusion 72 and the restriction protrusion 73 are biased toward one side of the observation protrusion portion 17 of the distal end configuration part 15 when the distal end cover main body 55 is attached to the distal end portion 8.

Thus, when the distal end cover main body 55 is attached to the distal end portion 8, the raising base 51 is arranged in a space formed between an inner wall of the distal end cover main body 55 and the second planar surface portion 17c of the observation protrusion portion 17, and the guide groove 51a of the raising base 51 faces the treatment instrument protrusion port 25.

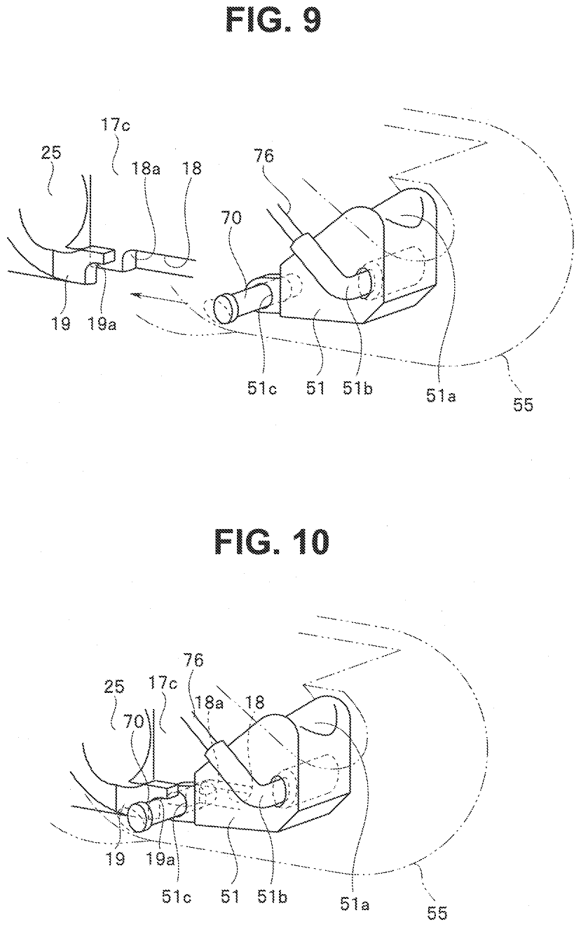

For example, as shown in FIGS. 9 and 13, a slide groove 18 capable of housing the case-side bearing protrusion 72 is formed in the second planar surface portion 17c formed on the observation protrusion portion 17 of the distal end portion 8. The slide groove 18 is formed in such a manner that a lower part of the second planar surface portion 17c is cut out in the direction of the insertion axis O. Then, a first distal end portion-side bearing portion 18a is formed at an end (proximal end) of the slide groove 18, as a bearing portion (support portion) having a partially circular arc abutting surface that faces the case-side bearing portion 72a.

The case-side bearing portion 72a and the first distal end portion-side bearing portion 18a are provided at positions in which one bearing hole is formed as a whole when the distal end cover main body 55 is attached to the distal end portion 8. Then, the first distal end portion-side bearing portion 18a abuts on a part, corresponding to the direction in which the raising base 51 rises (that is, for example, a part corresponding to a direction in which the raising base 51 is pulled by the operation wire 76 to be described mainly below (generally, an upper part of the shaft portion 70 in the present embodiment)), of the outer peripheral surface of the shaft portion 70, and can support the shaft portion 70.

In other words, the first distal end portion-side bearing portion 18a is set according to a direction of stress applied to the shaft portion 70 from the raising base 51 when rotating in the direction in which the raising base 51 rises (that is, a direction in which an advancing direction of the treatment instrument and the like is changed more significantly). In other words, the first distal end portion-side bearing portion 18a is provided, for example, at a position where a part of the outer peripheral surface of the shaft portion 70 approaching the treatment instrument protrusion port 25 can abut.

In addition, since the abutting surface of the first distal end portion-side bearing portion 18a is a partially circular arc surface formed in such a manner that a part of the second planar surface portion 17c is cut out (that is, a partially circular arc surface in which a lower part is opened), when the distal end cover main body 55 breaks up in the diameter expanding direction, the shaft portion 70 can move in a direction away from the first distal end portion-side bearing portion 18a.

Further, for example, as shown in FIGS. 5, 9, and 10, a distal end portion-side bearing protrusion 19 is provided on the end surface of the base portion 16 of the distal end configuration part 15, and a second distal end portion-side bearing portion 19a is provided at the distal end portion-side bearing protrusion 19, as a bearing portion (support portion) provided coaxially with the first distal end portion-side bearing portion 18a.

Here, the second distal end portion-side bearing portion 19a of the present embodiment includes a partially circular arc abutting surface formed in such a manner that a lower part of the distal end portion-side bearing protrusion 19 is cut out.

Thus, when the distal end cover main body 55 is attached to the distal end portion 8, the second distal end portion-side bearing portion 19a abuts on the part, corresponding to the direction in which the raising base 51 rises (that is, for example, the part corresponding to a direction in which the raising base 51 is pulled by the operation wire 76 to be described mainly below (generally, the upper part of the shaft portion 70 in the present embodiment)), of the outer peripheral surface of the shaft portion 70, and can support the shaft portion 70.

In other words, the second distal end portion-side bearing portion 19a is set according to the direction of stress applied to the shaft portion 70 from the raising base 51 when rotating in the direction in which the raising base 51 rises (that is, a direction in which an advancing direction of the treatment instrument and the like is changed more significantly). In other words, the second distal end portion-side bearing portion 19a is provided, for example, at the position where a part of the outer peripheral surface of the shaft portion 70 approaching the treatment instrument protrusion port 25 can abut.

In addition, since the abutting surface of the second distal end portion-side bearing portion 19a is a partially circular arc surface formed in such a manner that a part of the distal end portion-side bearing protrusion 19 is cut out (that is, a partially circular arc surface in which a lower part is opened), when the distal end cover main body 55 breaks up in the diameter expanding direction, the shaft portion 70 can move in a direction away from the second distal end portion-side bearing portion 19a.

For example, as shown in FIGS. 3 and 4, the wire mechanism portion 52 includes the guide sheath 75 and the operation wire 76 as a pulling member inserted into the guide sheath 75.

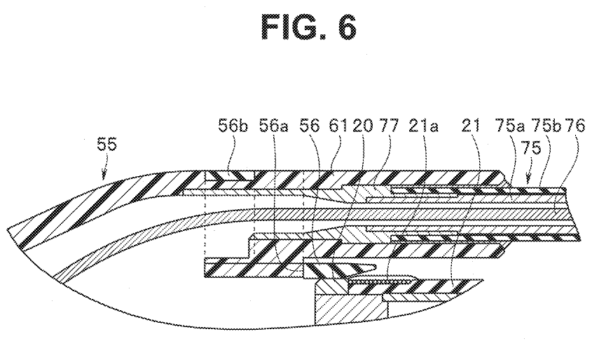

As shown in FIG. 6, the guide sheath 75 includes a coil 75a having flexibility and an envelope 75b that covers an outer periphery of the coil 75a.

A distal end pipe sleeve 77 is connected to a distal end of the guide sheath 75, the distal end pipe sleeve 77 fits into the sheath connection portion 61, and thus the guide sheath 75 is connected to the distal end cover main body 55.

Thus, the operation wire 76 inserted into the guide sheath 75 is introduced into the distal end cover main body 55, and the distal end of the operation wire 76 is connected to the wire connection portion 51b provided on the raising base 51.

On the other hand, a cylinder portion 78 is connected to a proximal end of the guide sheath 75 such that a wire shaft 79 is supported to be able to advance and retreat. A proximal end of the operation wire 76 is connected to a distal end of the wire shaft 79. In addition, a ring-shaped raising base operation member 79a is continuously provided on a proximal end side of the wire shaft 79, as an operation portion for the movement member.

Then, the wire shaft 79 advances and retreats through the operation of the raising base operation member 79a, and thus the raising base 51 can be displaced between a raised position and an inverted position.

According to such an embodiment, when the first and second distal end portion-side bearing portions 18a and 19a are provided on the distal end portion 8 (distal end configuration part 15) and the distal end cover main body 55 is attached to the distal end portion 8, the shaft portion 70 is supported by the first and second distal end portion-side bearing portions 18a and 19a to rotatably support the raising base 51 with respect to the distal end cover main body 55 (distal end cover 50), whereby the raising base 51 can be supported with sufficient support rigidity without an increase in diameter of the distal end portion 8.

In other words, when the distal end cover main body 55 is attached to the distal end portion 8, the shaft portion 70 is supported by the first and second distal end portion-side bearing portions 18a and 19a provided in the distal end configuration part 15 made of a rigid metal. Thus, even when the raising base 51 is provided on the distal end cover main body 55, the raising base 51 can be supported with sufficient support rigidity without measures to increase the thickness of the distal end cover main body 55, or the like.

In particular, when viewed from the shaft portion 70, the arrangement direction of the first and second distal end portion-side bearing portions 18a and 19a is a direction (approach direction to the treatment instrument protrusion port 25) in which the raising base 51 is pulled by the operation wire 76 when the raising base 51 is raised. Accordingly, in such a direction, a large stress acts on the shaft portion 70 when the raising base 51 rotates in the raising direction and thus the advancing direction of the treatment instrument is changed more significantly (compared with the case where the raising base 51 rotates in the inverted direction). Since the first and second distal end portion-side bearing portions 18a and 19a of the present embodiment support the shaft portion 70 in a direction in which such a large stress acts, even when the raising base 51 is provided on the distal end cover main body 55, the raising base 51 can be supported with sufficient support rigidity.

In such a case, when the abutting surfaces of the first and second distal end portion-side bearing portions 18a and 19a with the shaft portion 70 are formed in the partial arc shape by the cut-out and the distal end cover main body 55 breaks up due to the fragile portion 63 in the diameter expanding direction, the shaft portion 70 is formed to be movable in the direction away from the first and second distal end portion-side bearing portions 18a and 19a, and thus it is possible to prevent the break-up of the first and second distal end portion-side bearing portions 18a and 19a and the like due to the interference with the shaft portion 70 when the distal end cover 50 is removed from the distal end portion 8.

The operation wire 76 is connected to the distal end cover 50 (distal end cover main body 55) via the guide sheath 75, whereby the endoscope 2 is not necessary to include a drive shaft that rotates by the operation wire 76 and a seal member that seals the drive shaft and the like, the structure of the endoscope 2 can be simplified, and the endoscope 2 can be efficiently cleaned.

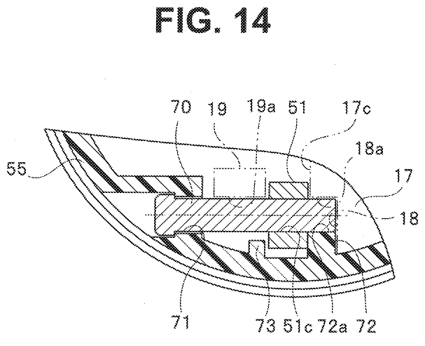

The configuration is described in the above-described embodiment as an example in which the case-side bearing hole 71 is sealed with the filling agent 74 in the state where the shaft portion 70 is inserted, but a configuration can also be adopted in which the case-side bearing hole 71 is not sealed with the filling agent 74 as shown in FIG. 14, for example. In FIG. 13, the shaft portion 70 is fixed to the distal end cover 50, and the raising base 51 is turnably supported with respect to the shaft portion 70. On the other hand, in FIG. 14, the shaft portion 70 and the raising base 51 are fixed, and the shaft portion 70 is turnably supported with respect to the distal end cover 50. Either of the configurations shown in FIGS. 13 and 14 may be used.

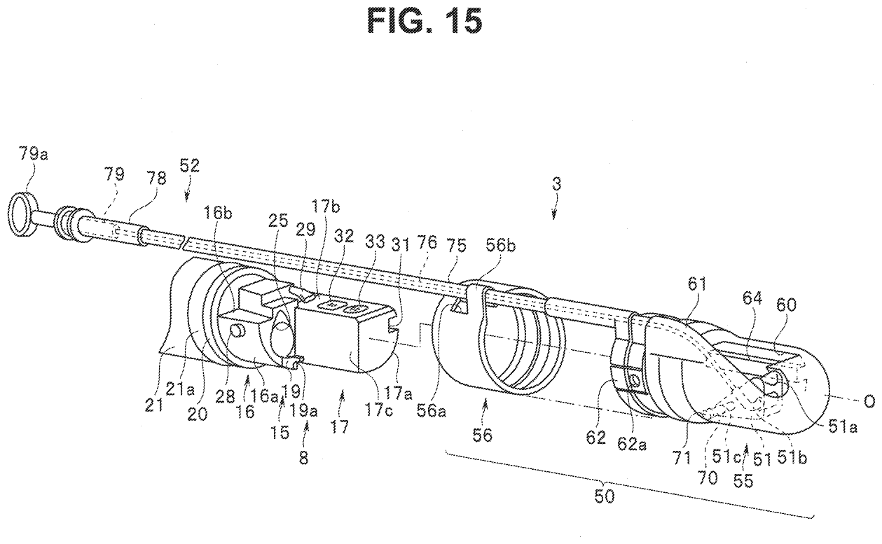

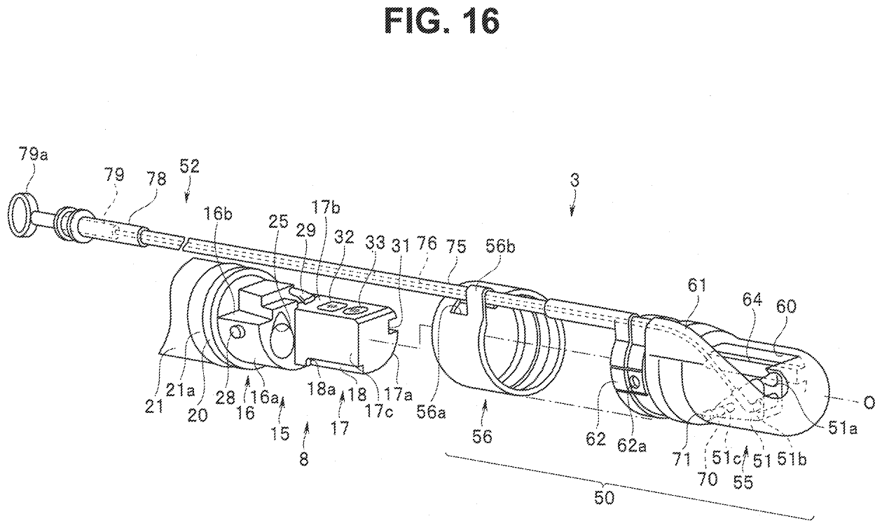

For example, as shown in FIGS. 15 and 16, a configuration can also be adopted as appropriate in which either the first distal end portion-side bearing portion 18a or the second distal end portion-side bearing portion 19a is not provided depending on the layout of the distal end configuration part 15, or the like.

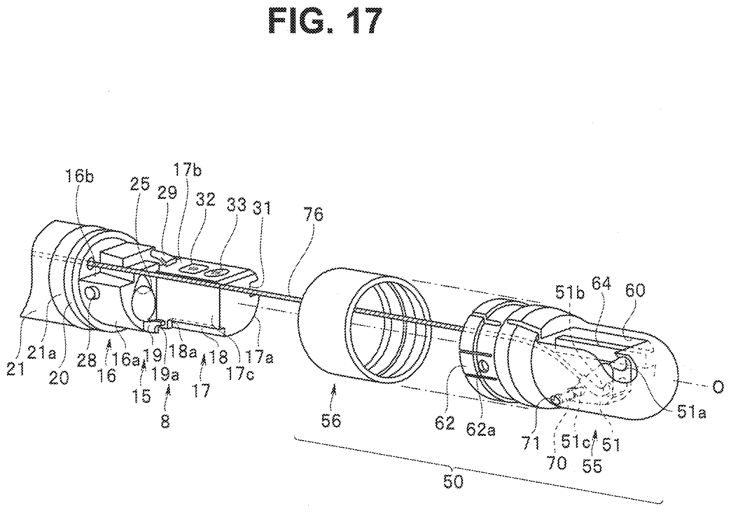

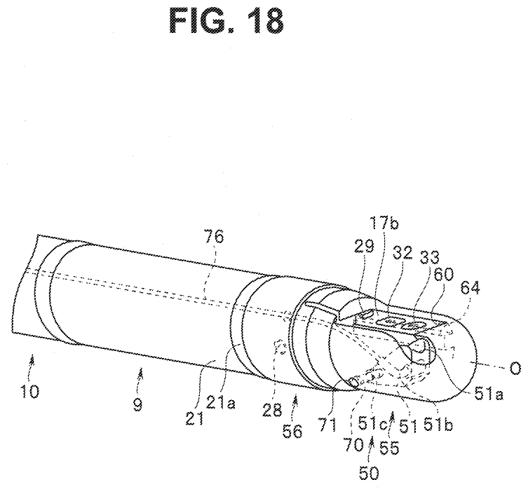

The configuration is described in the above-described embodiment as an example in which the operation wire 76 is inserted through the guide sheath 75 juxtaposed to the insertion portion 5, but a configuration can also be adopted in which the operation wire 76 is inserted into the insertion portion 5 as shown in FIGS. 17 and 18, for example.

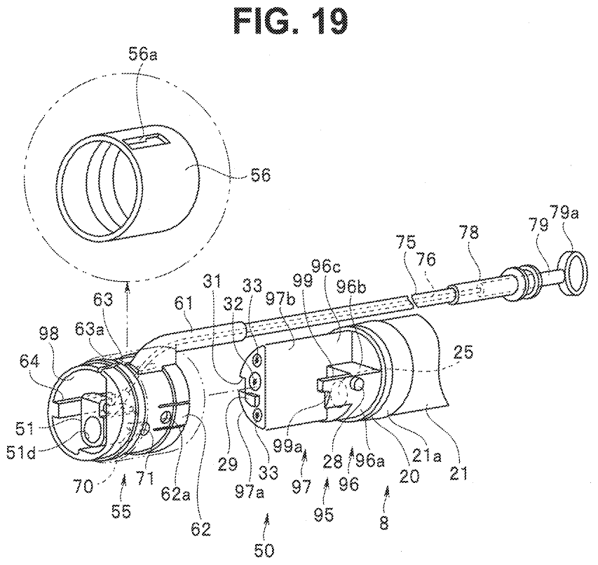

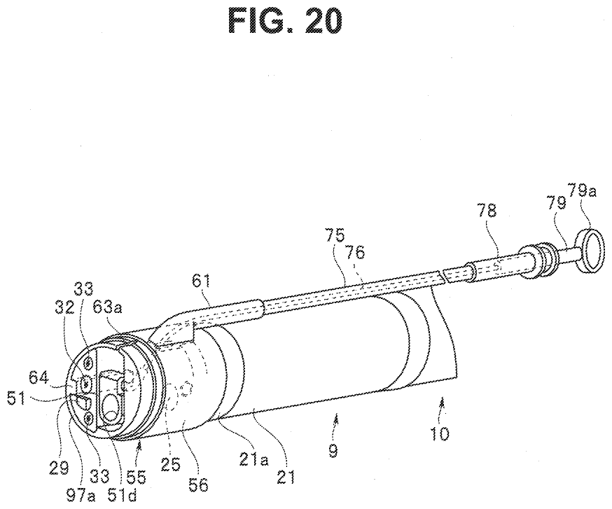

A second embodiment of the present invention will be described below with reference to FIGS. 19 to 21. A configuration of the present embodiment mainly differs, in that the distal end cover is applied to a so-called front-viewing endoscope 2 having a field of view in the direction of the insertion axis O, from the configuration of the above-described first embodiment in which the distal end cover device 3 is applied to the so-called side-viewing endoscope 2. The same components as the components in the first embodiment described above will be appropriately denoted by the same reference numerals and will not be described.

As shown in FIG. 19, a distal end configuration part 95 of the present embodiment includes a base portion 96 and an observation protrusion portion 97 protruding in the direction of the insertion axis O from a distal end of the base portion 96.

For example, as shown in FIG. 19, the base portion 96 is formed to have an approximate shape in which approximately a quarter of a cylinder is cut out. Thus, the base portion 96 includes a circular arc surface portion 96a, a first planar surface portion 96b, and a second planar surface portion 96c intersecting the first planar surface portion 96b on an outer periphery.

In addition, the observation protrusion portion 97 is formed to have an approximate shape in which substantially half of a cylinder arranged coaxially with the base portion 96 are cut out (that is, a substantially semi-cylindrical shape). Thus, the observation protrusion portion 97 includes, on an outer periphery, a circular arc surface portion 97a and a planar surface portion 97b parallel to the second planar surface portion 96c of the base portion 96.

On the end surface of the base portion 96, a treatment instrument protrusion port 25 is provided at a position adjacent to the planar surface portion 97b of the observation protrusion portion 97.

On the end surface of the observation protrusion portion 97, an observation window 32 of an image pickup unit not shown, an illumination window 33 of an illumination unit not shown, and a nozzle 29 configured to feed air or water toward the observation window 32 are provided. Then, with the observation window 32 being arranged in such a way, the endoscope 2 of the present embodiment is a so-called front-viewing endoscope having an observation field of view in the direction of the insertion axis O.

A distal end cover device 3 having substantially the same configuration as the configuration described in the above-described first embodiment can also be applied to a distal end portion 8 of the endoscope 2 in which the distal end configuration part 95 is configured in such a way. However, a distal end cover main body 55 of the present embodiment is provided with an opening portion 98 at a distal end rather than a lateral part of the distal end cover main body 55.

In addition, a raising base 51 of the present embodiment is provided with a guide hole 51d instead of the guide groove 51a.

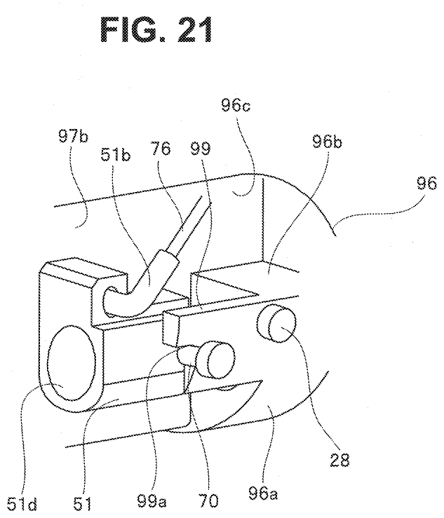

Further, for example, in the present embodiment as shown in FIGS. 19 and 21, a distal end portion-side bearing protrusion 99 is provided on the end surface of the base portion 96 of the distal end configuration part 95, and a third distal end portion-side bearing portion 99a is provided on the distal end portion-side bearing protrusion 99.

Here, the third distal end portion-side bearing portion 99a of the present embodiment includes a partially circular arc abutting surface formed in such a manner that a lower part of the distal end portion-side bearing protrusion 99 is cut out.

Thus, when the distal end cover main body 55 is attached to the distal end portion 8, the third distal end portion-side bearing portion 99a abuts on the part, corresponding to the direction in which the raising base 51 rises (that is, generally, the upper part of the shaft portion 70), of the outer peripheral surface of the shaft portion 70, and can support the shaft portion 70.

In other words, the third distal end portion-side bearing portion 99a is set according to the direction of stress applied to the shaft portion 70 from the raising base 51 when rotating in the direction in which the raising base 51 rises. In addition, since the abutting surface of the third distal end portion-side bearing portion 99a is a partially circular arc surface formed in such a manner that a part of the distal end portion-side bearing protrusion 99 is cut out (that is, a partially circular arc surface in which a lower part is opened), when the distal end cover main body 55 breaks up in the diameter expanding direction, the shaft portion 70 can move in a direction away from the third distal end portion-side bearing portion 99a.

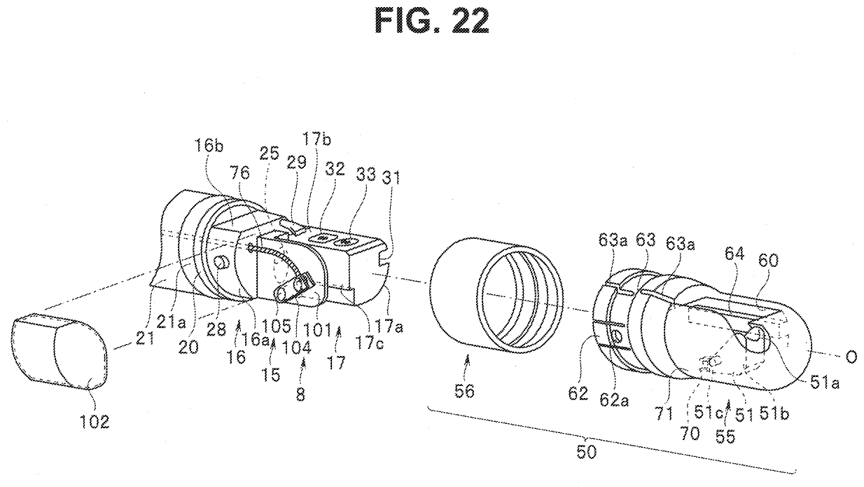

A third embodiment of the present invention will be described below with reference to FIGS. 22 to 25.

A configuration of the present embodiment mainly differs from the configuration of the first embodiment described above in that a wire mechanism portion is provided in the endoscope 2 and in terms of a configuration of a raising base and the like. The same components as the components in the first embodiment described above will be appropriately denoted by the same reference numerals and will not be described.

As shown in FIG. 22, the base portion 16 of the distal end configuration part 15 is provided with a protruding partition wall 101 that faces substantially parallel to the second planar surface portion 17c of the observation protrusion portion 17.

Further, an arm cover 102 is liquid-tightly attached to an outer surface side of the partition wall 101 (that is, a surface side not facing the second planar surface portion 17c), and an arm 104 is housed in an arm housing chamber 103 formed by the partition wall 101 and the arm cover 102.

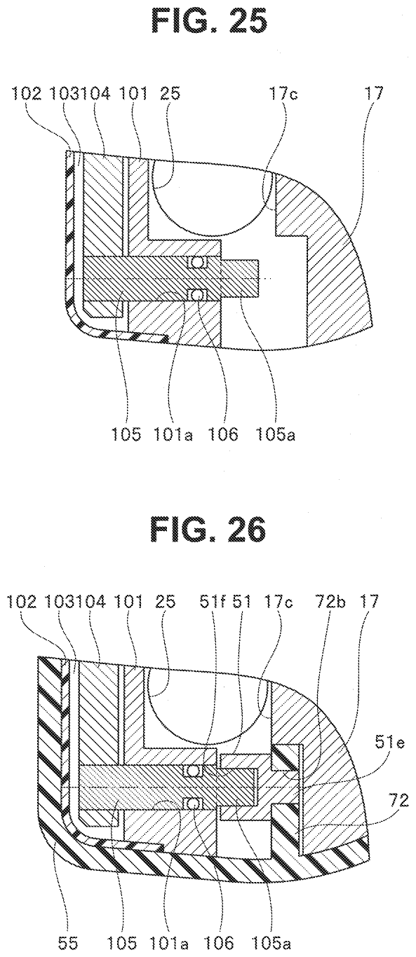

As shown in FIGS. 25 and 26, a through hole 101a is provided in the partition wall 101, and a drive shaft 105 is turnably supported in the through hole 101a. An O-ring 106 is provided on the drive shaft 105, and a space between the through hole 101a of the partition wall 101 and the drive shaft 105 is liquid-tightly sealed by the O-ring 106.

One end of the drive shaft 105 protrudes into the arm housing chamber 103 and is coupled to a fixed end side of the arm 104.

In addition, a distal end of the operation wire 76 inserted into the insertion portion 5 is led out into the arm housing chamber 103, and the operation wire 76 is coupled to a free end side of the arm 104.

Thus, the arm 104 can rotate the drive shaft 105 in conjunction with advancing/retreating movement of the operation wire 76.

Further, the other end side of the drive shaft 105 protrudes between the partition wall 101 and the observation protrusion portion 17. The other end portion of the drive shaft 105 protruding from the partition wall 101 is set as a fitting convex portion 105a including a plurality of fitting surfaces (for example, four fitting surfaces).

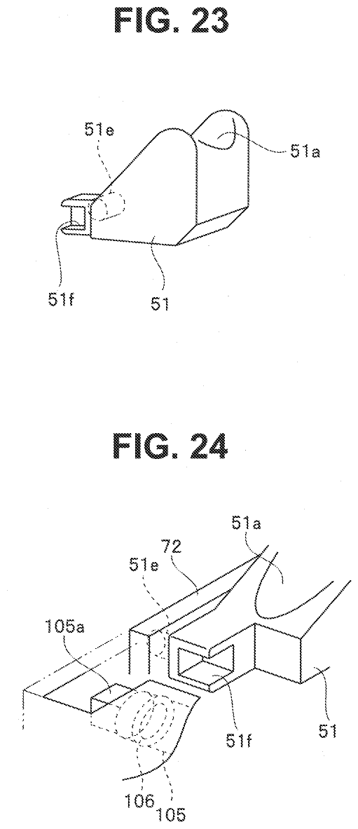

As shown in FIGS. 23 and 24, a shaft portion 51e as a shaft member protruding to the other side is integrally formed at a proximal end of the raising base 51 of the present embodiment. Then, the shaft portion 51e is supported by the distal end cover main body 55, and thus the raising base 51 is coupled to the distal end cover main body 55 in a swingable (displaceable) manner.

In other words, a case-side bearing protrusion 72 is provided inside the distal end cover main body 55, and a shaft portion 51e is supported by a case-side bearing hole 72b provided in the case-side bearing protrusion 72, whereby the raising base 51 is swingably supported with respect to the distal end cover main body 55.

On one side of the raising base 51, a fitting concave portion 51f, which can be fitted into the fitting convex portion 105a, is provided coaxially with the shaft portion 51e.

When the distal end cover main body 55 is attached to the distal end portion 8, the fitting convex portion 105a is fitted into the fitting concave portion 51f (see FIGS. 24 and 26), and thus the shaft portion 51e is supported by the case-side bearing hole 72b and is also supported by the drive shaft 105. In the present embodiment, as described above, the drive shaft 105 realizes the functions as a support portion.

Further, a driving force caused by the advancing/retreating movement of the operation wire 76 is transmitted to the shaft portion 51e (raising base 51) via the drive shaft 105, and the raising base 51 can swing between the raised position and the inverted position.

According to such an embodiment, even when the wire mechanism portion is provided in the endoscope 2, the same operational effects as the effects in the first embodiment described above can be obtained.

The present invention is not limited to the above-described embodiments, and various modifications and changes can be made within a technical scope of the present invention.

What is claimed is:

1. An insertion apparatus comprising:
an insertion portion configured to be inserted into a subject and configured to allow insertion of another insertion apparatus into the insertion portion;
a distal end cover detachably arranged at a distal end portion of the insertion portion;
a raising member provided on the distal end cover to change an advancing direction of the other insertion apparatus;
a shaft member provided on the distal end cover to rotatably support the raising member with respect to the distal end cover; and
a support portion provided at the distal end portion and configured to abut on the shaft member to support the shaft member when the distal end cover is attached to the distal end portion.

2. The insertion apparatus according to claim 1, wherein
the support portion includes an abutting surface configured to abut on a side in a predetermined direction of the shaft member.

3. The insertion apparatus according to claim 2, wherein
the abutting surface is provided in a direction in which a stress acting on the shaft member when the raising member rotates in a raising direction is applied.

4. The insertion apparatus according to claim 2, wherein
the distal end cover includes a fragile portion capable of breaking up when the distal end cover is removed from the distal end portion of the insertion portion, and
the shaft member is movable in a direction away from the abutting surface of the support portion when the fragile portion breaks up.

5. The insertion apparatus according to claim 4, wherein
the support portion is formed by being cut such that the shaft member is capable of being separated from the abutting surface in a direction opposite to the predetermined direction.

6. A distal end cover of an insertion apparatus, comprising:
a cover main body attached to a distal end portion of an insertion portion of the insertion apparatus;
a raising member provided on the cover main body to change an advancing direction of another insertion apparatus inserted into the insertion portion; and
a shaft member provided on the cover main body to rotatably support the raising member with respect to the cover main body, wherein
the shaft member is supported by a bearing provided at the distal end portion of the insertion portion of the insertion apparatus.
